(19) United States
(12) Patent Application Publication
(10) Pub. No.: US 2014/0336410 A1
(43) Pub. Date: Nov. 13, 2014

(54) ETHYLENICALLY UNSATURATED MONOMERS HAVING CHELATING FUNCTIONALITY

(71) Applicants: Dow Global Technologies LLC, Midland, MI (US); Rohm and Haas Company, Philadelphia, PA (US)

(72) Inventors: Scott Backer, Philadelphia, PA (US); Allen Bulick, Lansdale, PA (US); Joseph Manna, Quakertown, PA (US); Cynthia Rand, Sanford, MI (US); Jia Xie, Lake Jackson, TX (US)

(73) Assignees: ROHM AND HAAS COMPANY, Philadelphia, PA (US); DOW GLOBAL TECHNOLOGIES LLC, Midland, MI (US)

(21) Appl. No.: 14/354,613

(22) PCT Filed: Oct. 31, 2012
(86) PCT No.: PCT/US2012/062637

§ 371 Date: Apr. 28, 2014

Related U.S. Application Data
(60) Provisional application No. 61/553,574, filed on Oct. 31, 2011.

(51) Int. Cl. C07C 229/16; C07C 227/08

(52) U.S. Cl. CPC C07C 229/16; C07C 227/08
USPC 562/444

(57) ABSTRACT

The present invention provides novel polymerizable monomers having chelating functionality and processes to make them. In particular, the novel monomers are vinyl aminocarboxylates and are prepared by reacting iminodiacetic acid, iminodisuccinic acid, or a salt thereof and a vinyl epoxy benzene monomer.

### FIELD OF THE INVENTION

The present invention relates to novel polymerizable monomers having chelating functionality and methods to make them. In particular, the novel monomers are ethylenically unsaturated aminocarboxylates and are prepared by reacting iminodiacetic acid, iminodisuccinic acid, or a salt thereof, with a divinylbenzene monoepoxide.

### BACKGROUND OF THE INVENTION

Synthetic detergents typically consist of a dispersant, a builder, and other miscellaneous ingredients such as brighteners, perfumes, anti-redeposition agents and enzymes. The dispersant typically comprises a surfactant and functions to separate dirt, soil and stains from fabric and other substrates. Polyacrylates are well known and commonly used dispersant compounds. The builder binds with and forms a complex with metal cations, such as calcium and magnesium ions found in “hard water,” which otherwise interfere with the dispersant activity. Such binding and complex formation is also commonly referred to as “chelating” and compounds capable of such interaction with metal ions are known as “chelating agents.” Phosphates are excellent chelating agents, which is why they were historically used as builders for detergents. However, even after wastewater treatment, large amounts of phosphorus found their way into streams, rivers, lakes and estuaries. In natural water bodies, phosphorous acts as a fertilizer, increasing growth of algae and aquatic weeds, which depletes the amount of oxygen available for healthy fish and aquatic life, whose numbers then decrease. Consequently, most jurisdictions have limited or banned the use of phosphates in detergents.

In the search for phosphate substitutes, amino carboxylate compounds have been found to be effective chelating agents and, therefore, useful as builders for laundry and automatic dishwashing detergents. For example, U.S. Pat. No. 3,331,773, teaches preparation of water soluble polymers having chelating functionality by grafting water soluble chelating monomers onto water soluble polymers. Diethylenetriamine, ethylenediamine tetraacetic acid, and other polyalkylene polyamine polyacetic acids are identified as examples of chelating monomers suitable for grafting onto water soluble polymers.

U.S. Pat. No. 5,514,732 also describes contact lenses made from water insoluble polymers having chelating functionality. The polymers are made from aminopolycarboxylic acids with a polymerizable olefinic group, as well as a hydrophilic monomer and one or more crosslinking monomer.

U.S. Patent Application No. 2008/00262192 describes an water-soluble polymer having a high chelating performance and clay dispersancy which is made by polymerizing an amino group-containing allyl monomer derived from adding an amine compound, such as iminodiacetic acid (IDA), to an allyl monomer, such as allyl glycidal ether (AGE). Also according to U.S. Patent Application No. 2008/00262192, the amino group-containing allyl monomer may be polymerized with other polymerizable monomers including, without limitation, unsaturated monocarboxylic acid monomers.

U.S. Patent Application No. 2009/0082242 discloses a phosphate free dish washing liquor comprising exfoliated nanoclay, a clay-dispersing polymer, as well as other components including known chelating agents such as nitrilotriacetates (NTA), ethylene diamine tetra acetate (EDTA), propylene diamine tetraacetic acid, (PDTA), ethylene diamine N,N′-disuccinic acid (EDDS) and methyl glycine di-acetic acid (MGDA), or their salts.

The present invention provides novel polymerizable monomer compounds which have chelating functionality, as well as polymers made therefrom which shall be useful in aqueous systems for scale inhibition, soil removal, tea destaining, particulate dispersion and metal ion binding.

### SUMMARY OF THE INVENTION

The present invention is an ethylenically unsaturated aminocarboxylate monomer having one or more of the following structures:

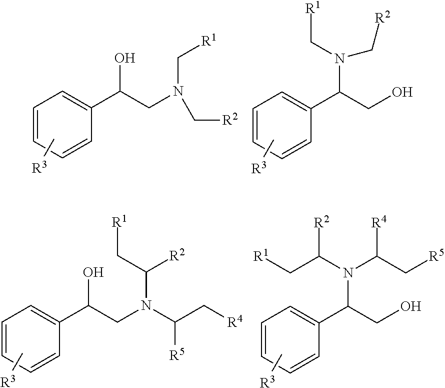

wherein R¹ is COOX¹, R² is COOX², R⁴ is COOX⁴ and R⁵ is COOX⁵; X¹, X², X³, X⁴, X⁵, are each, independently, hydrogen or a mono- or polyvalent cation and the total charge on the monomer is zero; and R³ is a polymerizable ethylenically unsaturated group located at the ortho-, para-, or meta-substituted position of the benzene ring. For example, R³ may be —CH═CH₂. The mono- or polyvalent cation may be selected from the group consisting of: Na⁺, K⁺, NH₄⁺, organic ammonium ions, Ca²⁺ and MG²⁺.

The present invention also provides a process for preparing the vinyl aminocarboxylate monomers, comprising reacting iminodiacetic acid, iminodisuccinic acid, or a salt thereof, with a divinylbenzene monoepoxide.

### DETAILED DESCRIPTION OF THE INVENTION

All percentages stated herein are weight percentages (wt %), unless otherwise indicated.

Temperatures are in degrees Celsius (° C.), and ambient temperature means between 20 and 25° C., unless specified otherwise.

“Polymerizable” as used to described a monomer or other molecule means that the monomer or other molecule has at least one carbon-carbon double bond and is capable of forming additional covalent bonds with other monomers or molecules of its kind, other polymerizable monomers or molecules, or polymers having polymerizable pendant groups, under normal polymerization conditions, and become incorporated in to the product polymer.

As used herein, the term “(meth)acrylic” includes acrylic acid and methacrylic acid.

As used herein, the term “(meth)acrylates” includes esters of acrylic acid and esters of methacrylic acid.

The present invention relates to new monomer compositions which are polymerizable monomers having chelating functionality and are referred to hereinafter as “ethylenically unsaturated aminocarboxylate monomers.” The ethylenically unsaturated aminocarboxylate monomers of the present invention may have one or more of the following structures:

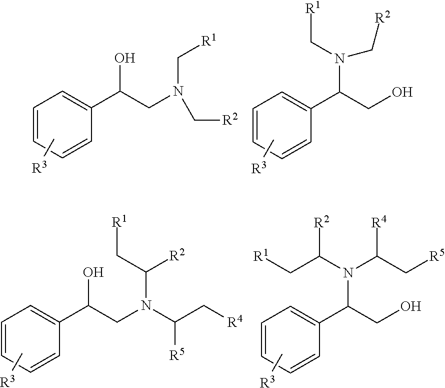

wherein R¹ is COOX¹, R² is COOX², R⁴ is COOX⁴ and R⁵ is COOX⁵; X¹, X², X³, X⁴, X⁵, are each, independently, hydrogen or a mono- or polyvalent cation and the total charge on the monomer is zero; and R³ is a polymerizable ethylenically unsaturated group located at the ortho-, para-, or meta- substituted position of the benzene ring. For example, R³ may be —CH═CH₂.

In some embodiments, for example, X¹ and X² are each, independently, a mono- or polyvalent cation selected from the group consisting of: Na⁺, K⁺, NH₄⁺, organic ammonium ions, Ca²⁺ and Mg²⁺.

The present invention also provides a process for making the vinyl aminocarboxylate monomers which comprises reacting, in the presence of a phase transfer catalyst, iminodiacetic acid (IDA) or iminodisuccinic acid (IDS), with a divinylbenzene monoepoxide (DVBMO) monomer having the following structure:

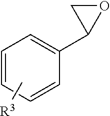

wherein R³ is a polymerizable vinyl (—HC═CH₂) group located at the ortho-, para-, or meta- substituted position of the benzene ring. Hereinafter, abbreviations for the possible structures of DVBMO in the ortho, para, and meta positions are o-DVBMO, p-DVBMO, and m- DVBMO. Note that “(o-, p-, m-)DVBMO” means one or more of the o-DVBMO, p-DVBMO, and m-DVBMO.

The iminodiacetic acid (IDA), iminodisuccinic acid (IDS), or salt thereof, and (o-, p-, m-)DVBMO may be reacted in any suitable ratio, as is readily determinable by persons of ordinary skill. The process for making the vinyl aminocarboxylate in accordance with the present invention may be conducted at ambient temperatures. The foregoing process may be performed at a pH between 4 and 14, for example without limitation between 7 and 14.

The phase transfer catalyst is not particularly limited and various phase transfer catalysts useful for the above-described reaction are known to persons of ordinary skill in the relevant art. For example, without limitation, suitable phase transfer catalysts include benzyltrimethylammonium chloride, tetra-n-butylammonium bromide, methyltrioctylammonium chloride, hexadecyltributylphosphonium bromide, dimethyldiphenylphosphonium iodide, and methyltriphenoxyphosphonium iodide.

For example, where (o-, p-, m-)DVBMO is provided for reaction with IDA, the reaction as demonstrated by the following reaction equation:

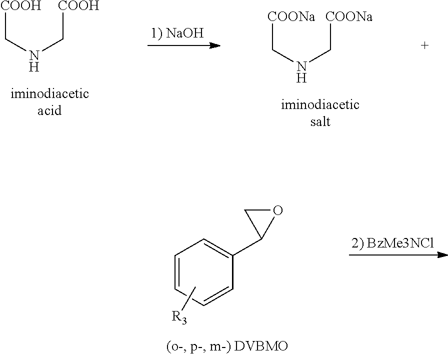

The foregoing reaction proceeds via opening of the epoxy ring and attachment of the IDA functional group to one of the carbon atoms of the opened epoxy ring. Thus, the ethylenically unsaturated aminocarboxylate monomers resulting from the foregoing reaction (IDA+(o-, p-, m-)DVBMO) will have one or more of the following possible structures:

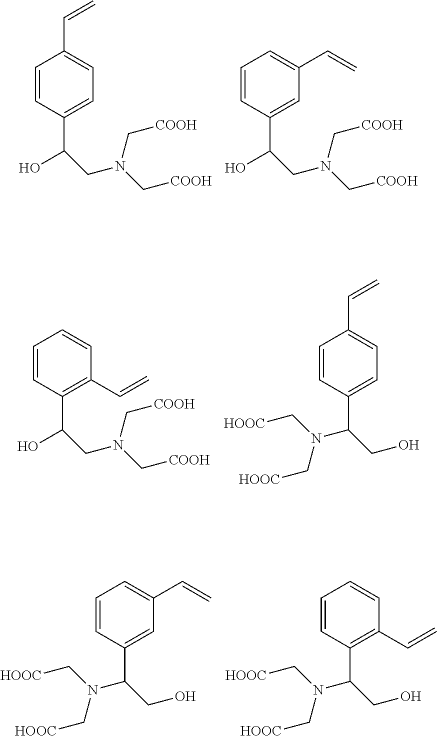 [SMILES: C=Cc1ccc(C(O)CN(CC(=O)O)CC(=O)O)cc1 ; C=Cc1cccc(C(O)CN(CC(=O)O)CC(=O)O)c1 ; C=Cc1ccccc1C(O)CN(CC(=O)O)CC(=O)O ; C=Cc1ccc(C(CO)N(CC(=O)O)CC(=O)O)cc1 ; C=Cc1cccc(C(CO)N(CC(=O)O)CC(=O)O)c1 ; C=Cc1ccccc1C(CO)N(CC(=O)O)CC(=O)O]

Of course, as will be recognized by persons of ordinary skill, the ethylenically unsaturated aminocarboxylate monomers of the present invention may be in their acidic form, as shown above, or they may be salts thereof, wherein one or more hydrogen atoms has been substituted for a mono- or polyvalent cation. The mono- or polyvalent cations may be, for example, selected from the group consisting of: Na⁺, K⁺, NH₄⁺, organic ammonium ions, Ca²⁺ and Mg²⁺.

As will be recognized by persons of ordinary skill in the relevant art, where ethylenically unsaturated aminocarboxylate monomers of the present invention are produced by reaction of iminodisuccinic acid (IDS) with (o-, p-, m-) DVBMO, multiple isomers will be present in the product mixture, similar to those shown above for the IDA-(o-, p-, m-) DVBMO reaction products.

The use, application and benefits of the present invention will be clarified by the following discussion and description of an exemplary embodiment of the present invention.

### EXAMPLES

### Synthesis of IDA-(p)-DVBMO

To a 500 mL round bottom flask equipped with a magnetic stir bar and an addition funnel, 198 mL of Deionized water is added. The water is placed in an ice bath, and set to stir at a minimum of 300 rpm. Iminodiacetic acid (66.55g) is added to the stirring water to form a slurry. 80 g of 50wt % sodium hydroxide is slowly added to the slurry, and after approximately 20 minutes, the iminodiacetic acid is fully solubilized. 1.86 g of a phase transfer catalyst (Benzyltrimethylammonium chloride) is charged to the vessel and allowed to dissolve completely over approximately five minutes. During this time, 73.1 grams of (p)-DVBMO is charged to the addition funnel. The (p)-DVBMO is added drop wise to the stirring reaction mass, and when complete, allowed to stir at room temperature until the reaction mass transitions from two phases to a single phase. This is determined by visual observation, in which prior to completion, the reaction mass is hazy, and would separate into two distinct phases upon termination of stirring. Upon completion, the reaction mass is observed to be a clear solution, which is stable upon termination of stirring. This solution is stable to storage under ambient conditions and can be used as such.

In certain cases, solid monomer is required. To produce it from the above solution, sulfuric acid is added drop wise while stirring in order to adjust the pH of the solution, halting the flow of sulfuric acid when the pH is between 7-7.5. The solution was placed in an ice bath to promote crystallization. The crystals are isolated via filtration using a Buchner funnel and allowed to dry overnight before storage.

We claim:

1. An ethylenically unsaturated aminocarboxylate monomer having one or more of the following structures: 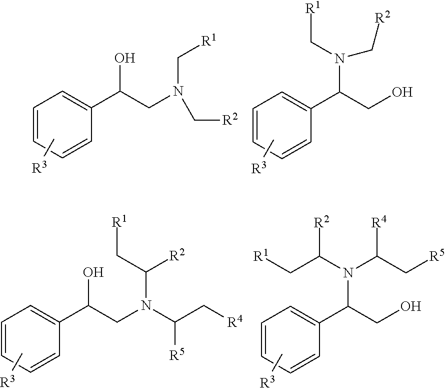
wherein R¹ is COOX¹, R² is COOX², R⁴ is COOX⁴ and R⁵ is COOX⁵; X¹, X², X³, X⁴, X⁵, are each, independently, hydrogen or a mono- or polyvalent cation and the total charge on the monomer is zero; and R³ is a polymerizable ethylenically unsaturated group located at the ortho-, para-, or meta- substituted position of the benzene ring.

2. The ethylenically unsaturated aminocarboxylate monomer according to claim 1, wherein R³ is —CH═CH₂.

3. The ethylenically unsaturated aminocarboxylate monomer according to claim 1, wherein the mono- or polyvalent cation is selected from the group consisting of: Na⁺, K⁺, NH₄⁺, organic ammonium ions, Ca²⁺ and Mg²⁺.

4. A process for preparing the ethylenically unsaturated aminocarboxylate monomer according to claim 1, comprising reacting iminodiacetic acid, iminodisuccinic acid, or a salt thereof, with divinylbenzene monoepoxide.

5. The process according to claim 4, wherein said reacting step occurs in the presence of a phase transfer catalyst.
